(19) United States
(12) Patent Application Publication
(10) Pub. No.: US 2021/0060588 A1
(43) Pub. Date: Mar. 4, 2021

(54) ROTATABLE DIFFUSER DEVICE

(71) Applicant: Keith Wootan, Layton, UT (US)

(72) Inventor: Keith Wootan, Layton, UT (US)

(21) Appl. No.: 17/011,862

(22) Filed: Sep. 3, 2020

Related U.S. Application Data
(60) Provisional application No. 62/895,425, filed on Sep. 3, 2019.

(51) Int. Cl. B05B 3/08; A61L 9/14

(52) U.S. Cl. CPC B05B 3/08; A61L 2209/13; A61L 9/14

(57) ABSTRACT

A diffuser that diffuses a liquid and projects atomized particles into air from a housing that is detachable from a rotatable base plate. The base of the diffuser is configured with a motor for rotating the base plate and with circuitry for providing power to components in the housing. Rotation of the base plate causes rotation of the housing vent during use. Control circuitry in the base can operably control properties of the diffuser components and the vapor being dispersed by the diffuser.

### CROSS-REFERENCE TO RELATED APPLICATIONS

This application claims the benefit of and priority to U.S. Provisional Patent Application Ser. No. 62/895,425, filed on Sep. 3, 2019, entitled “ROTATABLE DIFFUSER DEVICE,” which application is expressly incorporated herein by reference in its entirety.

### BACKGROUND

Essential oils have long been known and used to provide soothing and healing natural scents. These oils are often used in conjunction with diffusers that break down the oil into microscopic particles and project the scented oil particles into the air. Typical diffusers use heat, ultrasonic vibration waves, fans, or nebulizers to break down the oils into atomized particles. The atomized particles are dispersed into the air by the vaporizer as a mist or vapor.

In some instances, a diffuser can be picked up and rotated to a desired location/position to control the directionality in which the vapor is dispersed. This is a relatively convenient way to reposition a diffuser, if the diffuser is battery powered. However, when the diffuser is powered by a cord, it can be cumbersome to position the diffuser in a desired orientation without exposing the cord in an undesired manner.

To allow a user to adjust the direction in which the atomized particles are dispersed, some diffusers are also configured with a vent on a manually rotatable lid or cap, which the user can manually rotate (e.g., similar to twisting a lid on or off a bottle), without requiring the entire diffuser to be rotated in order to change the direction in which the vapor is dispersed. In such implementations, it is possible to manually rotate a diffuser vent without having to worry about the direction that the power cord is situated, if there is one.

Typical diffusers often experience problems with the buildup of oil residue that collects over time. Oils are highly viscous by nature and often form a gum-like substance in cracks or grooves through which the oils pass. In instances where the vent can be manually rotated relative to the rest of the housing, the connections between the vent and the rest of the diffuser components can accumulate this gooey/gum-like oil residue buildup. This can also make it difficult to rotate the vented cap/lid on the diffuser, which can be annoying and functionally limiting.

The gum-like substance requires the diffuser to be cleaned regularly whenever possible; however, certain diffusers cannot be disassembled. Consequently, cleaning the oil residue becomes impossible. Failure to clean the gummy substance prevents the diffuser from functioning optimally and impacts the efficacy of the scent as leftover residue from one oil may contaminate the scent of the oil that is being diffused.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

The disclosed embodiments are generally directed to diffuser devices that are configured with both a base and a diffuser housing that is physically and electrically detachable from the base. The base includes a stationary base portion, a rotatable base plate, and control circuitry that controls rotation of the rotatable base plate. Power is transferred to the diffuser housing through the rotatable base plate. In some embodiments, the control circuitry is also operable to control diffuser components within the diffuser housing.

In some instances, the rotatable base plate has positioning components used for centering and retaining the diffuser housing in one or more removable positions on the rotatable base plate with fixed positioning relative to the rotatable base plate while the rotatable base plate rotates relative to the rest of the base.

The diffuser housing houses diffuser components used for atomizing and projecting the atomized particles through a vent or nozzle port that is adjustable and that is operable to be rotated with the diffuser housing during operation and rotation of the base plate of the diffuser base. The vent/nozzle is also configured, in some embodiments, to be adjusted independently and relative to the diffuser housing.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 is a perspective view of a diffuser device comprising a diffuser housing and a base, with a rotatable base plate positioned between the base and the diffuser housing.

FIG. 2A and FIG. 2B show a perspective view of a diffuser device including an adjustable nozzle.

FIG. 3A is a partial perspective side view of a diffuser device and includes electrical and physical coupling components and a motor for driving rotation of a base plate through a slip ring.

FIG. 3B is a side view of the diffuser of FIG. 3A.

FIG. 4A illustrates a base portion for a diffuser, the base portion having a rotatable/rotating base plate that rotates radially about a central axis passing through the base portion that remains relatively stationary to the rotating base plate during use.

FIG. 4B illustrates a diffuser housing with a bottom surface that is configured in size and shape to conform to the top surface of the rotatable base plate of the diffuser housing shown in FIG. 4A.

FIG. 5 illustrates another configuration of a diffuser device with a diffuser housing and a base having a rotatable base plate, the bottom surface of the diffuser housing being configured in size and shape to be physically coupled in a secure position to the top surface of the rotatable base plate during use.

FIG. 6 illustrates another configuration of a diffuser device with a diffuser housing and a base having a rotatable base plate, the bottom surface of the diffuser housing being configured in size and shape to be physically coupled in a secure position to the top surface of the rotatable base plate during use.

### DETAILED DESCRIPTION

The disclosed embodiments generally relate to a type of vaporizer or diffuser apparatus capable of vaporizing a liquid and diffusing the vapor into the surrounding environment/atmosphere.

In some embodiments, the diffuser includes a base and a diffuser housing. The base comprises a stationary base portion and a rotating/rotatable base plate. The rotating/rotatable base plate is physically and electrically coupled to the stationary base portion and is structured to rotate radially about a central axis that passes through the center of the base and that is coaxially aligned with a central axis passing through the center of the diffuser housing.

The diffuser housing is physically and electrically coupled to the rotating/rotatable base plate during use and is operable to be selectively detached from the rotatable base plate by lifting the diffuser housing up away from the base plate.

Additionally, the diffuser includes (i) some diffuser components provided to diffuse a liquid into atomized particles and (ii) a vent/nozzle that rotates with the diffuser housing and that provides a throughway through which the atomized particles can escape the diffuser to enter the environment.

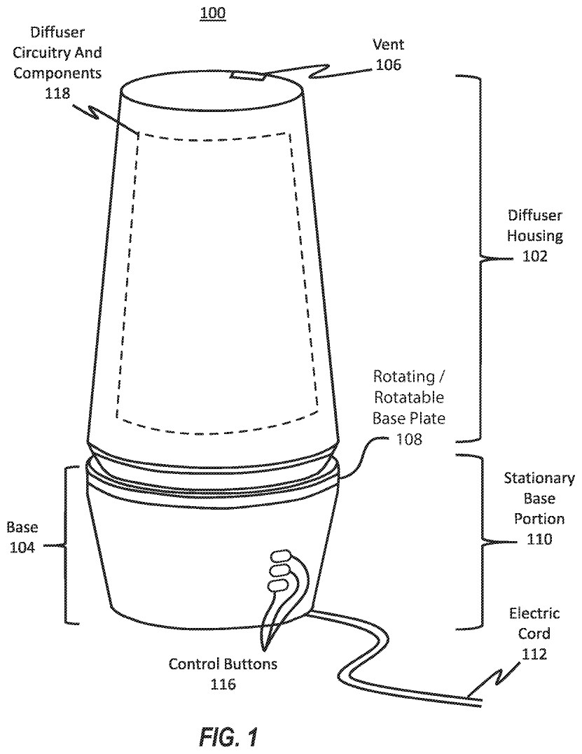

Referring to the drawings, FIG. 1 shows an embodiment of a diffuser device 100 comprising a diffuser housing 102 and a base 104. A rotatable (or rotating) base plate 108 is positioned between the diffuser housing 102 and the base 104. In some embodiments, the base includes a rotatable/rotating base plate 108.

The rotatable base plate 108 is considered one of the structural components or structural features of the base 104. The rotatable base plate 108 is also referred to herein as a “rotating base plate” or simply as a “base plate,” even though it may or may not be in a state of rotating at any given moment.

The diffuser housing 102 and base 104 may be formed out of a variety of materials including plastic, metal, wood, rubber, or any combination of those materials and may be formed to any shape or size. The diffuser housing 102 and base 104 are generally rigid to maintain their shapes and to offer some protection against spills or outside elements such as humidity, heat, or cold. The diameter of the diffuser housing 102 and base 104, measured at a point of the greatest distance/diameter of each, is preferably within a range of about two inches and five inches, with a taper of between one half an inch to a full inch. However, in some instances, the diameter of the diffuser housing 102 and base 104 is less than two inches or more than five inches.

In the illustrated embodiment, the diffuser housing 102 and base 104 are formed as generally cylindrical shapes, being positioned in co-axial alignment, including a central axis 130 of the diffuser housing 102 that is in co-axially alignment with the central axis 135 of the base 104 when the diffuser housing 102 is positioned on and connected with the base plate 108.

In some embodiments the base 104 increases in width/diameter from the bottom of the base 104 to the top of the base 104 and the diffuser housing 102 decreases in width/diameter from the bottom of the diffuser housing 102 to the top of the diffuser housing 102. The taper in the base 104 and diffuser housing 102, defined by the increases/decreases in diameters may be more than a half an inch or even a full inch along the full length of the base 104 and/or diffuser housing 102.

It will be appreciated that the tapered diameter design is only one configuration of the diffuser housing 102 and base 104. Accordingly, the diffuser housing 102 and base 104 may also comprise other designs and shapes, such as a rectilinear, oval, spherical, pyramidal, or any other shape and can include varying widths and heights, which may be tapered or un-tapered.

As shown, the diffuser housing 102 includes a vent 106 through which diffused or vaporized oil is dispersed into the air. In some embodiments, the vent 106 is rotatable (along with the diffuser housing 102) as the diffuser housing 102 rotates in sync with the rotatable base plate 108 that it is detachably positioned on. In some instances, as described in more detail below, the vent, diffuser housing 102 and rotatable base plate 108 are rotated automatically by a power source and control circuitry.

The diffuser housing 102 can be configured to remain in a fixed position on the base plate 108 during rotation of the base plate 108, which may rotate at any predetermined rate or frequency. This causes the diffuser housing 102 to rotate radially about the central axis 130 of the diffuser housing 102 as the base plate correspondingly rotates radially about the central axis of the base.

It is noted that higher frequencies/speeds of rotation tend to throw/disperse the vapor that is being expelled from the diffuser housing 102 into farther (i.e. more remote) areas of the environment (relative to the current location of the diffuser housing 102) while lower frequencies tend to disperse the vapor into closer areas of the environment (relative to the location of the diffuser housing 102). Example rotation frequencies include, but are not limited to, 0.1 revolutions per second (RPS), 0.2 RPS 0.25 RPS, 0.3 RPS, 0.4 RPS, 0.5 RPS, 0.75 RPS, 1.0 RPS, 2.0 RPS, and sometimes even more than 2.0 RPS.

The disclosed embodiments represent improvements over existing diffuser devices that have manually or mechanically adjustable vented caps (which are configured to rotate relative to the rest of the diffuser housing), because the vents and components in such traditional diffusers can get gummed up due to excess oil accumulating in grooves of the cap. In contrast, the disclosed embodiments enable the vent to be rotated with the entire diffuser housing and without requiring a specialized cap or other mechanical components/configurations of the diffuser housing to enable rotation of the vent. Likewise, it is not necessary to manually pick up and rotate the diffuser or diffuser housing 102 to rotate the vent 106 to effectively distribute the vapor a full 360° or another desired range surrounding the diffuser.

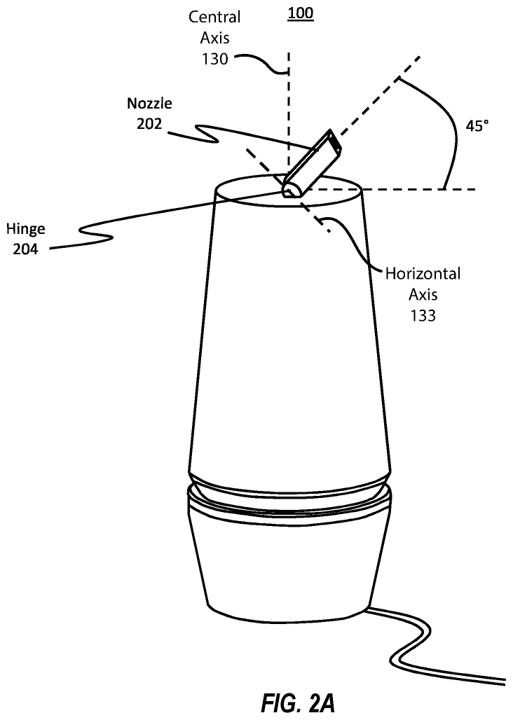

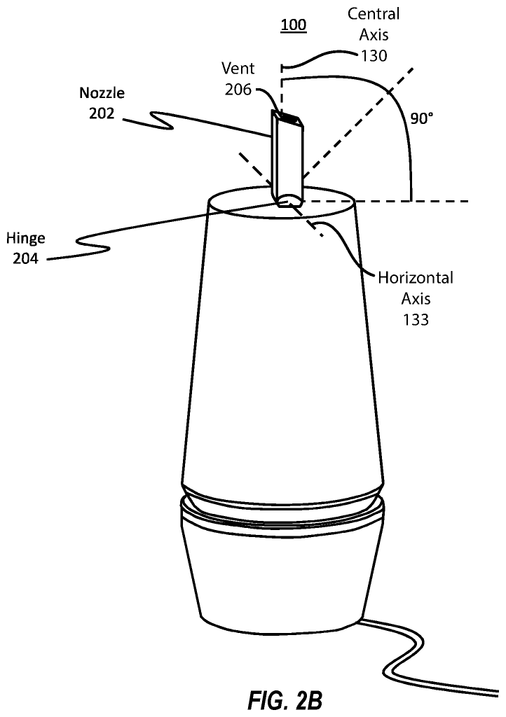

In some embodiments, as shown in FIG. 2A and FIG. 2B, the vent 206 is incorporated into or replaced by a nozzle 202. The nozzle 202 is vertically adjustable (either manually or electromechanically) about a horizontal axis that runs perpendicular to the orientation of the central axis of the diffuser.

As shown in FIG. 2A, the vented nozzle 202 may be attached to the diffuser housing using a hinge 204 that is rotatable about a horizontal axis 133. In FIG. 2A, the nozzle 202 is positioned at approximately 45 degrees from a horizontal plane associated with the horizontal axis 133 and the atomized particles will be projected at this angle, or in an upward diagonal direction.

In FIG. 2B, the nozzle 202 is adjusted vertically to about 90 degrees from the horizontal plane associated with the horizontal axis 133 and in direct alignment with the central axis 130. In this configuration, the dispersion of the atomized particles expelled from the diffuser is approximately straight up (e.g., relative to the gravity direction). With the currently illustrated embodiment, the nozzle 202 is adjustable to any customizable angle within the range of 90 degrees to about 10 degrees from the horizontal plane/horizontal axis 133.

In some embodiments, the vented nozzle is attached to a mechanical lever (not presently shown, but which are known to those of skill in the art) within the diffuser housing 102 that is powered by an electro-mechanical motor/gear device to adjust the vent/nozzle angle for dispersing the vapor.

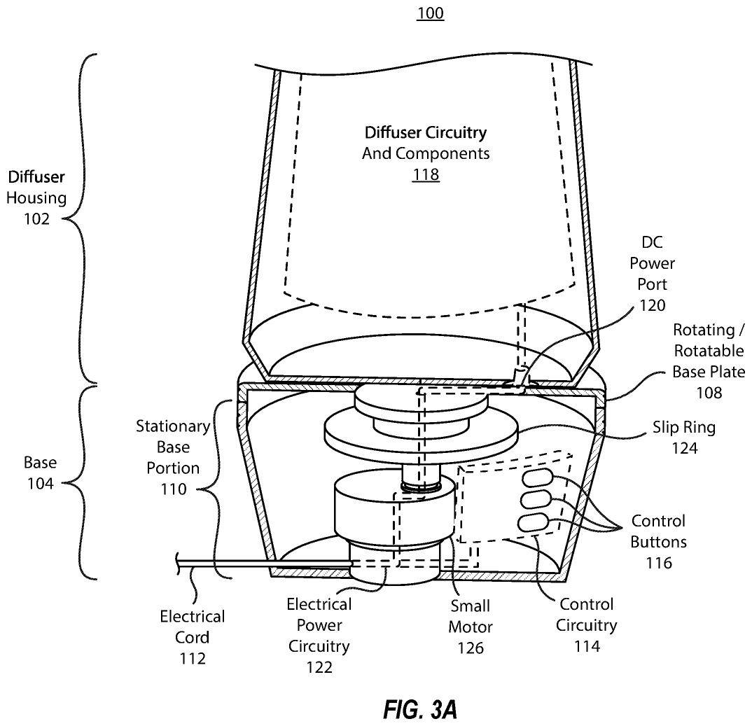

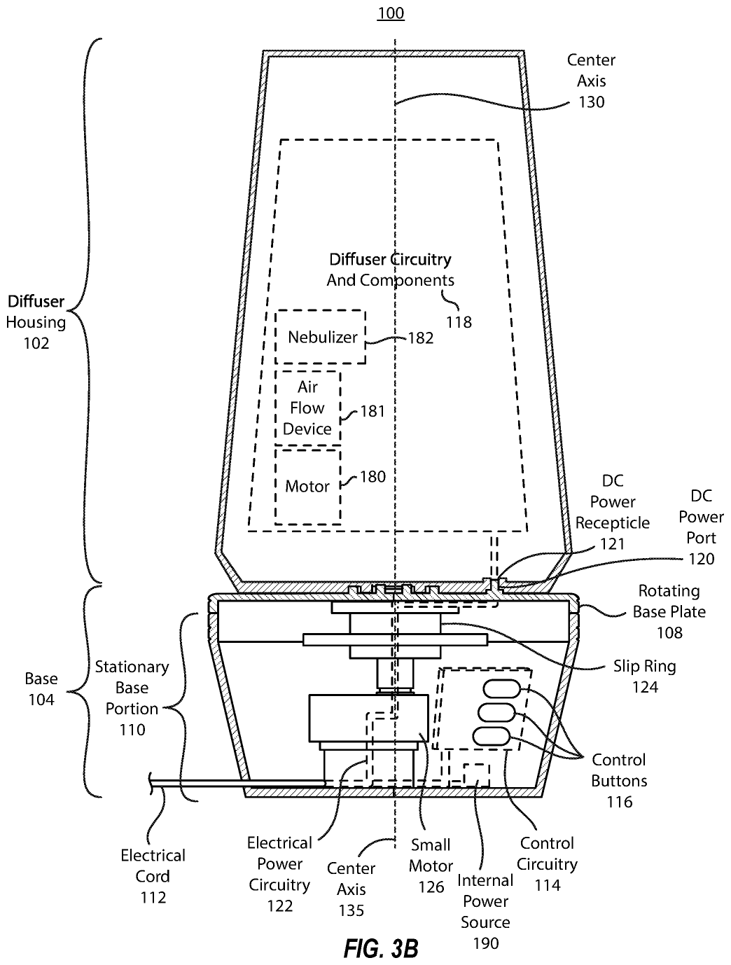

Attention is now directed to FIG. 3A, which illustrates a cutaway perspective view of an example diffuser 100 that includes a diffuser housing 102 and base 104. FIG. 3B is also described at the same time, which illustrates a cutaway side view of the example diffuser 100.

As shown, an electric cord 112 is used with the diffuser 100 to provide electricity to the base 104 from a power source and to all of the powered components in the diffuser (e.g., the base components, the rotatable base plate, and the diffuser housing components) through one or more electrical connections.

In some embodiments, the electric cord 112 is replaced by an internal power source 190, such as removable and/or rechargeable battery power supplies.

In some embodiments, the electrical cord 112 receives electric power from an electric outlet (not pictured) or another power source. The electric power travels from the outlet to the electric cord 112 and through electrical power circuitry 122 (e.g., wires, traces, and other circuitry that is abstractly illustrated in the figures). For instance, the electrical power circuitry 122 electrically/conductively couples the components within the base 104, including the control circuitry 114 and control buttons 116, as well as to the diffuser housing 102 and diffuser circuitry and components 118. It will be appreciated that the diffuser can operate off of either AC power or DC power. When configured as an AC device, the diffuser can receive power directly from the electrical outlet. When configured as a DC device, the diffuser may include an AC/DC adapter as part of the illustrated control circuitry 114 and/or power circuitry 122.

In some embodiments, the electric cord 102 provides an electrical current used in combination with a small motor 126 and a slip ring 124 to electrically couple the base 104 through the rotatable base plate 108 to the diffuser housing 102 and circuitry contained therein. Such motors and slip rings, are known to those of skill in the art and do not need to be further described at this time. Importantly, however, it is noted that the slip ring 124 enables power/current to pass through the slip ring 124 while a portion of the slip ring is radially rotated relative to the rest of the slip ring 124, and such that the slip ring 124 can be used with the small motor 126 to power rotation of the top portion of the slip ring 124 that is connected to the base plate 108 and to cause rotation of the base plate 108, relative to the stationary base portion 110 of the base 104 and while still supplying power up through the slip ring 124 and into/through a DC power port 120 that is electrically connected to the diffuser circuitry/components 118 within the diffuser.

The electrical power circuitry 122 illustrated is one example of conductive traces, lines, wires and/or other circuitry that may be used to pass current/power through the base 104, through the slip ring 124, and through/along the rotatable base plate 108 to a DC power port 120 that is exposed on the top surface of the base plate.

The current embodiment also illustrates user interface control buttons 116 that are operable, when pressed/selected by a user, for controlling and interfacing with the control circuitry 114 to control different settings to selectively make adjustments related to the operation of the motor 126, and other components, and so as to provide selectable control over the rotation of the rotatable base plate 108.

For example, one or more of the control buttons 116 may be pressed to cause the control circuitry 114 to adjust the maximum and minimum angle(s) of rotation for base plate 108, the speed(s) of rotation, intermittent and/or continuous periods of rotation of the base plate.

One or more of the control buttons 116 can also be selected to cause the control circuitry to adjust/control other components, such as the nebulizer(s), heater(s), pump(s), fan(s), nozzle motor(s), and other components (not shown but included within the diffuser circuitry and components 118), for controlling the volume of vapor that is created and dispersed from the diffuser housing, as well as the magnitude and directionality of propulsion for propelling the atomized particles.

The control buttons 116 can be any type of hard key (e.g., actual physical buttons disposed on the outer portion of the diffuser) or they can be soft keys (e.g., a user interface key displayed on a user interface).

In some embodiments, the control circuitry 114 is also connected to sensors (e.g., temperature/humidity sensors, as well as proximity/light sensors) that are operable in conjunction with settings set by the control buttons 116 and, as controlled by the control circuitry 114, for dynamically and automatically adjusting different power/control settings of the different motors and diffuser circuitry for adjusting properties of the vapor being produced and/or how it is dispersed and the directionality of dispersing the vapor based on sensor readings (e.g., based on the temperature and/or humidity sensor reading(s) and/or other environmental conditions detected by any combination of the sensors).

The control circuitry 114 is configured to adjust power settings and/or to send control signals through the electrical power circuitry to one or more of the diffuser circuitry components (e.g., motor 180, air flow device 181, nebulizer 182 and/or any other component in the diffuser housing 102), as well as to any components in the base 104, such as the small motor 126.

Some settings of the control circuitry 114 include, for example, controlling the speed, angle, and period/frequency of rotating the base plate. For example, the diffuser components within the diffuser housing 102 would rotate with the diffuser housing 102 along with the rotating base plate 108 to a certain degree and then stop, intermittently, and then rotate back to the original angle and pause intermittently, before rotating back again.

In some embodiments, the diffuser can operate in accordance with a programmed rotation frequency. For example, the programmed rotation frequency may initially cause the diffuser to rotate at a relatively higher rate upon initial startup (e.g., to quickly disperse the vapor) and then progressively reduce the rotation rate. In some embodiments, the programmed rotation frequency can periodically be increased and then subsequently decreased. Any type of rotation programming may be used.

The control buttons 116 also allow for a setting that can vary based on the environment or surroundings. In one embodiment, different degrees of rotation may be preferred depending on where the diffuser device 100 is placed. For example, if the diffuser device 100 is placed in the middle of a room, it may be desired to set the diffuser to rotate the diffuser housing 102 of the device 360 degrees continuously. Alternatively, if a diffuser is placed in a corner, it may be desired to set the diffuser to rotate the diffuser housing 102 of the device within a range of 90 degrees so that the diffused oil is not being vented directly into the wall.

In some embodiments, the control circuitry (as controlled through the settings selected with the control buttons, or based on default settings) affect the speed, period, timing, rotational angle, and other properties of the rotation of the diffuser housing via the rotation of the base plate according to personal preference. The settings can also control the properties of the vapor being produced, as well as the vertical positioning of the vent (e.g., when the vented nozzle is connected to electromechanical motors).

It will be appreciated that the disclosed embodiments enable a user to customize and adjust the direction that the diffused oil/liquid is dispersed by the disclosed diffuser.

The settings for controlling the diffuser, such as for controlling the settings of the control circuitry and corresponding components in the diffuser housing and the base, including the motor that rotates the rotating base, are input through the control buttons 116, for example, and may be stored in storage components (e.g., flash memory, RAM, ROM, or other memory) contained within the control circuitry and/or connected to the control circuitry (although not presently shown) and that are executed by one or more processors included in the control circuitry to implement/cause the functionality that is described.

In some instances, the control buttons are physical and visible buttons that are and manually operable and positioned on the base plate. In other embodiments, the control buttons are display buttons that are rendered on an interface display on the diffuser.

In other embodiments, the control buttons are virtual buttons that are not visible on the base but are, instead, presented on a user interface that is displayed on a mobile device or computing system that is wirelessly connected to the control circuitry 114. Although three buttons are shown, different quantity of buttons may also be used, each associated with different functionality (e.g., one for power, one for control of the base, such as rotation control of the rotatable/rotating base plate 108, one for control of the components within the diffuser housing 102, such as the nebulizer 182, air flow device 181 and/or motor 180).

Notably, in some embodiments, the control buttons are replaced by or supplemented with remote interface features that are built into the control circuitry and which enable a user to control the operation of the diffuser wirelessly and without even having to touch the diffuser (e.g., via use of a smart device). In such embodiments, the user can monitor and control the diffuser settings without even being in the same room.

To facilitate such remote access and control, a user interface for controlling the control circuitry 114, not shown, will present a virtual representation of the diffuser and positioning of the vent within a virtual representation of the room/environment it is placed in. The user interface will also provide a display of different settings of the diffuser, such as position of the vent, properties of the vapor being produced and/or other settings described herein for the diffuser. Then, a wireless input control device such as a wireless remote control or mobile phone (with the corresponding interface) is used to provide the input signals to the control circuitry for causing the control circuitry to adjust the diffuser settings.

In some cases, the diffuser is an Internet-of-Things (IoT) device that can be connected to other IoT devices located within a home, office, or other building. In some embodiments, the diffuser can then be controlled by a smart thermostat sensor or climate control device.

The control circuitry, with settings set by the control buttons, not only control rotation settings of the base plate, as mentioned above, but can also control all of the electrically powered components of the diffuser.

The power circuitry (line) shown in FIGS. 3A-3B include one or more wires, or electrical traces/contacts, that collectively comprise a communication means for sending control signals to the powered components, as well as power for powering the components. In some instances, different electrical lines are used to transmit power from those that are used to transmit control instructions and/or sensor readings.

As noted above, FIG. 3A-3B generally illustrates how a slip ring rotor may be used to rotate the rotatable base plate while allowing power to pass through the slip ring from the stationary base portion 110 of the base 104 to the rotating base plate 108 and correspondingly on to the diffuser housing 102 positioned on the base plate 108.

It will be appreciated, however, that other embodiments may also be used to rotate the base plate. For example, in some embodiments, a squirrel-cage rotor is used instead of a slip ring rotor. Additionally, another embodiment may utilize a wound-rotor motor with variable resistance. Other motor variations known by persons skilled in the art are also able to be incorporated in the electrical power circuitry 122. It is worthwhile to note that in these embodiments, the base 104 is powered and capable of rotating the base plate whether the diffuser housing is attached or detached to the base plate.

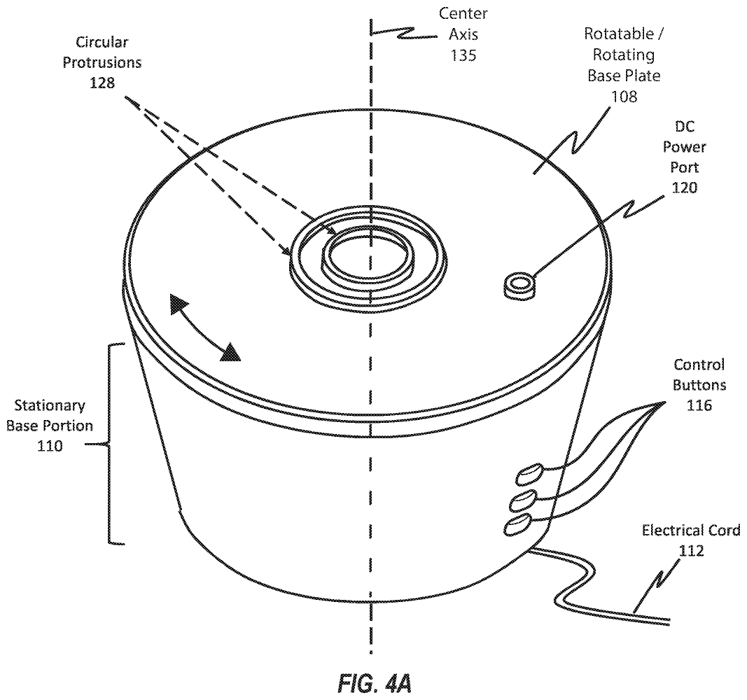

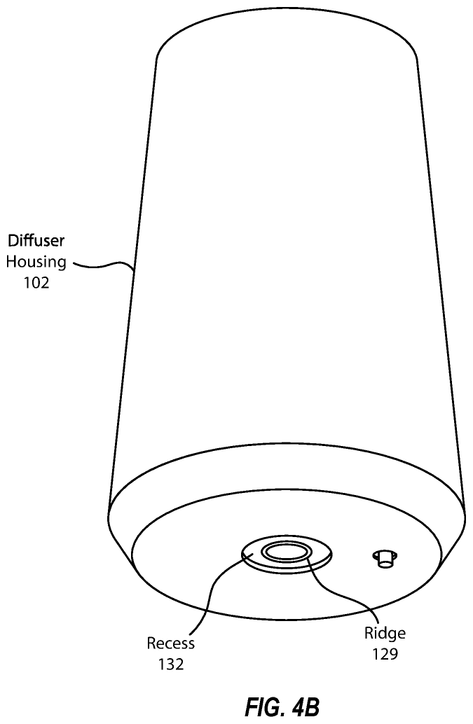

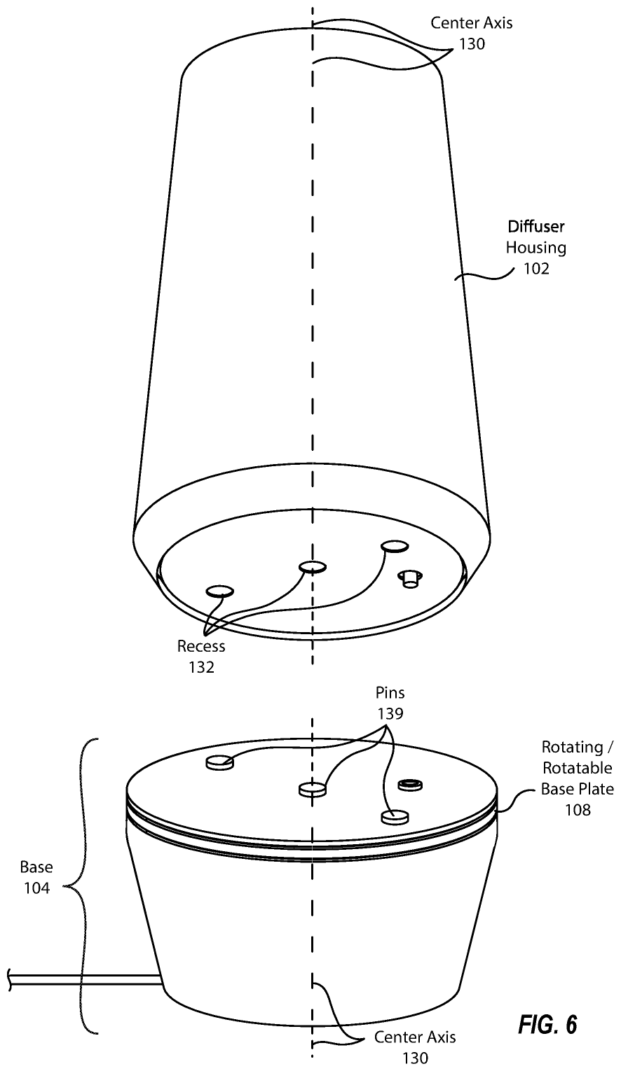

In some embodiments, a DC power port 120 is provided to electrically and physically couple the diffuser housing to the base via the base plate. In some instances, the DC power port 120 includes a male connection where the male connection protrudes from the rotatable base plate 108 and is received by a female connection aperture/power receptacle 121 in the diffuser housing 102. In other embodiments, the DC power port 120 includes a female connection aperture being part of the rotatable base plate 108 and the diffuser housing 102 has a mating male connector protruding from the bottom of the diffuser housing 102 (e.g., FIGS. 4A, 6).

Although the current embodiments show electrical power being provided from an electrical cord 112, such as may be plugged into an electric outlet (not shown), alternative embodiments include other electrical power sources. Some of these include and are not limited to USB connection, removable batteries, induction power, or built-in rechargeable batteries that are contained within the stationary base portion 110 and are charged either through an electrical cord, 112, USB, a wireless charging station, or any other means that is generally known in the art.

The DC power port 120 is one example of a means for supplying power to the diffuser housing. In other embodiments, the diffuser housing 102 includes a rechargeable battery that is used for powering the components in the diffuser housing 102. In this embodiment, the rechargeable battery is charged through induction from an induction plate in the base or base plate.

In another embodiment, a rechargeable battery in the diffuser housing 102 is charged using one or more surface contacts (not presently shown) that allow power to be transmitted from the rotatable base plate 108 to the rechargeable battery in the diffuser housing 102 when the diffuser housing is positioned on the rotatable base plate.

Additionally, although the power circuitry is shown as extending from the control circuitry 114 up to the diffuser housing, it may be appreciated that there may be one or multiple connections, including surface contacts, that are able to maintain electrical connectivity and conductivity during rotation of the rotating base plate 108.

The diffuser device embodiments described include diffuser housing 102 that houses diffuser circuitry and components 118 located within the diffuser housing 102. The diffuser circuitry and components 118 utilize power supplied from the base 104 to atomize and diffuse the particles and project the particles in the air.

In some instances, the DC power port 120 is a means through which the diffuser circuitry and components 118 receive that power from the base. For example, in the case of a heat diffuser, power from the base 104 is provided to the diffuser circuitry and components 118 to provide energy to heating elements which are then able to atomize the oil contents. With an ultrasonic diffuser, energy from the base 104 is used to create the ultrasonic vibration waves that atomize the oil elements. Another common diffuser includes nebulizers that utilize the power provided from the base 104 to break down and nebulize the oils into atomized particles. Fans are often used in combination to atomize and project the atomized oils into the air. These components and other necessary diffuser circuitry and components 118 draw power through the DC power port 120 from the base 104 of the diffuser device 100.

In some embodiments, the DC power port 120 also provides some structural support to ensure that the diffuser housing 102 is properly positioned on the base plate, such as by being substantially centered (e.g., co-axially aligned along a central axis 135 passing through the center of the base 104, which is also co-axially aligned with the central axis 120 passing through the diffuser housing 102 when the diffuser housing 102 is positioned on the rotating base plate 108) and to physically retain the diffuser housing on the rotating base plate 108 during use/rotation.

Other mechanisms can also be provided in addition and/or alternatively to further control positioning and retaining of the diffuser housing on the base plate. Some examples of these other mechanisms are shown in FIGS. 4A-6, for example.

In FIG. 4A, for example, the rotatable base plate 108 includes two circular rings, ridges or protrusions 128 that protrude up and away from the top surface of the base plate 108 and that are used for centering and retaining the diffuser housing 102 on the rotatable base plate 108. The bottom of the diffuser housing 102 also includes a circular opening/recess 132 and a corresponding ridge 129 that fits between the ridges 128. Alternatively, grooves can be configured in the base of the diffuser housing (not shown) that correspond and physically engage the structural protrusions of the rotatable base plate 108.

It is noted that the circular openings 132 and ridges/protrusions 128, 129 help to ensure the centering and retaining of the diffuser housing 102 central axis 130 to be co-axially aligned with the central axis 132 of the base when positioned on the base plate 108.

In another embodiment, the circular protrusions 128 are located on the underside of the diffuser housing 102 and the opening 132 and ridge 129 are located on the rotatable base plate 108, in opposite configurations (not shown).

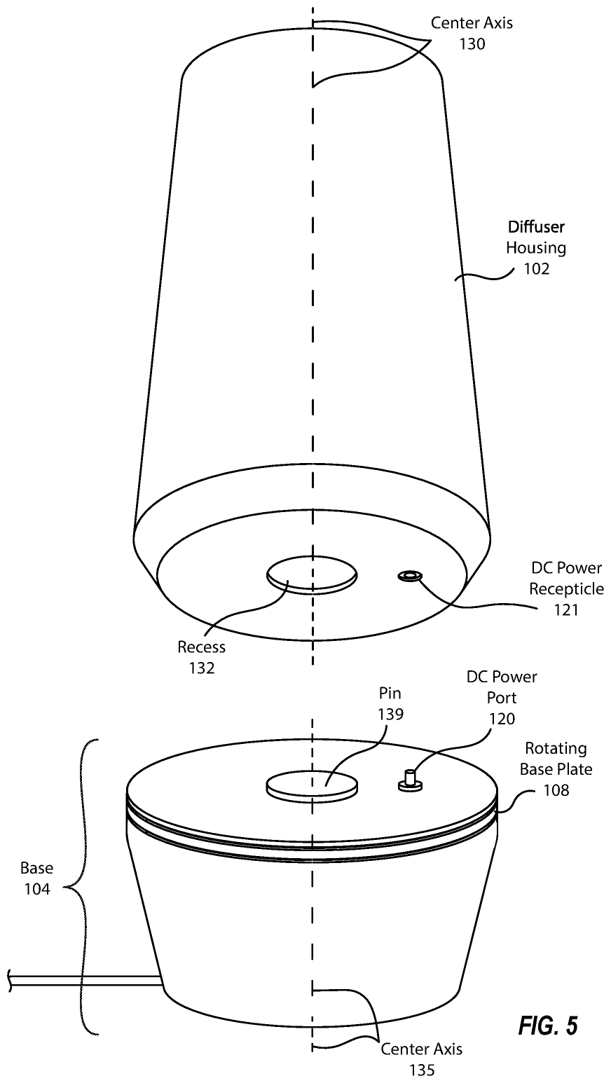

In another embodiment, shown in FIG. 5, a recess 132 is formed into the bottom of the diffuser housing 102 that is configured in size and shape to accommodate a pin 139 or protrusion formed into the top of the base plate 108.

In another embodiment, shown in FIG. 6, multiple pins 139 in the rotatable base plate 108 are protruding from the base plate 108 and are sized to fit within a corresponding set of recesses 132 formed into the bottom of the diffuser housing 102. In an alternative configuration, the pins may be formed along the base of the diffuser housing and be configured to fit within recesses formed into the base plate (not presently illustrated).

In another embodiment, not presently shown, the rotatable base plate 108 may be configured with magnets that interact with magnets of opposing polarity that are positioned within the diffuser housing, and to help secure the diffuser housing in a desired position on the base plate during use.

In another embodiment, the bottom of the diffuser housing 102 includes an adhesive fabric or other material, such as a hook and loop material (e.g., Velcro), that physically adheres to or attaches to a corresponding material/fabric that is positioned on the rotatable base plate 108

Other embodiments also include configuring the rotatable base plate 108 and/or housing surface with a nonslip textured plastic or rubber that provides a friction engagement to help retain the diffuser housing 102 in place and such that the center axis 130 of the diffuser housing 102 is co-axially aligned with the center axis 132 of the base 104.

Accordingly, it will be appreciated that the disclosed embodiments generally relate to an improved type of diffuser apparatus. This apparatus beneficially reduces (or even entirely eliminates) the undesired buildup of gum-like impeding material in the diffuser (particularly between components in the diffuser that would be used to enable rotation of a portion of the diffuser vent/housing). Instead, the rotational movement for the diffuser is external to the diffuser housing and is configured in the base of the diffuser, by using a rotatable base plate connected to the diffuser with a slip ring, for example.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A diffuser device that diffuses a liquid and projects the diffused liquid into air, the diffuser device comprising:
a base including:
a stationary base portion; and
a rotatable base plate physically and electrically coupled to the stationary base portion, the rotatable base plate arranged to rotate about a central axis extending through the stationary base portion; and
a diffuser housing that is physically and electrically coupled to and detachable from the rotatable base plate, the diffuser housing including:
diffuser components for diffusing a liquid into atomized particles, the diffuser components being housed within the diffuser housing; and
a vent that rotates with a rotation of the diffuser housing, the vent being a passageway through which atomized particles are projected into the air.

2. The diffuser device of claim 1, wherein the base includes control circuitry that controls the diffuser components located within the diffuser housing and rotatable movement of the rotatable base plate.

3. The diffuser device of claim 2, wherein the rotatable base plate rotates between 0 and 360 degrees during use in response to a control setting of the control circuitry.

4. The diffuser device of claim 3, wherein the rotatable base plate rotates at a variable speed controlled by control circuitry in the diffuser.

5. The diffuser device of claim 3, wherein the rotatable base plate rotates 360 degrees continuously.

6. The diffuser device of claim 3, wherein the rotatable base plate is operable to rotate in two different rotational directions during use.

7. The diffuser device of claim 1, wherein the vent is vertically adjustable.

8. The diffuser device of claim 1, wherein the electrical coupling in the base includes a motor and a slip ring whereby electricity is supplied through the base plate from the base to the diffuser housing.

9. The diffuser device of claim 1, wherein the base has an independent power source and is independently controllable even when the diffuser housing is detached from the rotatable base plate.

10. A diffuser device that diffuses a liquid and projects the diffused liquid into air, the diffuser device comprising:
a base including:
a stationary base portion;
a rotatable base plate; and
a slip ring that physically and electrically couples the stationary base portion to the rotatable base plate; and
a diffuser housing that is physically and electrically detachably coupled to the rotatable base plate, the diffuser housing including:
diffuser components for diffusing a liquid into atomized particles, the diffuser components being housed within the diffuser housing; and
a vent that rotates with a rotation of the diffuser housing, the vent being a passageway through which atomized particles are projected into the air.

11. The diffuser device of claim 10, wherein the slip ring electrically transmits power to a diffuser power port from the base to the diffuser housing via an electric port formed in the top of the rotatable base plate.

12. The diffuser device of claim 10, wherein the diffuser device further includes a first motor for driving rotation of the rotatable base plate, which is located within the base portion, and a second motor for operating the diffuser component for diffusing the liquid into atomized particles.
